(19) United States
(12) Patent Application Publication
(10) Pub. No.: US 2026/0215779 A1
(43) Pub. Date: Jul. 30, 2026

(54) SHUNT DEVICE

(71) Applicant: The Cleveland Clinic Foundation, Cleveland, OH (US)

(72) Inventors: Jose Luis NAVIA, Cleveland, OH (US); Paresh M. VASANDANI, Cleveland, OH (US); Jorge BALZAN, Cleveland, OH (US); Torey HOVEST, Cleveland, OH (US); Lucas HARDER, Cleveland, OH (US); Bo CLAYMORE, Cleveland, OH (US)

(21) Appl. No.: 19/143,491

(22) PCT Filed: Dec. 28, 2023
(86) PCT No.: PCT/US2023/086266

§ 371 Date: Jun. 26, 2025

Related U.S. Application Data
(60) Provisional application No. 63/435,613, filed on Dec. 28, 2022.

(51) Int. Cl. A61B 17/11

(52) U.S. Cl. CPC A61B 17/11; A61B 2017/1107; A61B 2017/1139

(57) ABSTRACT

A shunt device is manipulable between a collapsed configuration and a shunt configuration. The shunt device includes a shunt frame assembly having a first anchor and a second anchor that are configured to radially flare from the collapsed configuration toward the shunt configuration. The second anchor defines a proximal opening of the shunt device in the shunt configuration, and the first anchor and second anchor define a common central axis. The shunt device further includes a flow director that extends distally from the shunt frame assembly and defines a fluid passageway and a distal opening at an end of the fluid passageway. The flow director is configured to redirect at least some fluid flowing through the fluid passageway such that the at least some fluid is discharged through the distal opening in a fluid direction transverse to the central axis.

### CROSS-REFERENCE TO RELATED APPLICATIONS

This application claims the benefit of U.S. provisional application Ser. No. 63/435,613 filed Dec. 28, 2022, the contents of which are incorporated by reference.

### TECHNICAL FIELD

The present disclosure relates to a shunt device and more particularly, a shunt device that is useful for treatment of pulmonary artery hypertension.

### BACKGROUND

Pulmonary hypertension (PH) is a disease characterized by a sustained increase in pulmonary artery pressure. Generally, patients with an average pulmonary artery pressure of 25 mmHg or higher are considered to have PH or have symptoms of PH. It is estimated that up to 50-70 million individuals, almost 1% of all people, are affected by PH worldwide. PH is classified into 5 groups, with the most common form of PH being Group 2, which is PH due to left heart disease. Left heart disease can include heart failure (both with preserved and reduced ejection fractions), as well as valvular disorders, that lead to the heart not being able to adequately pump blood out of the heart. These issues lead to blood backing up into the left atrium and subsequently the lungs, which raises the pressure in the lungs.

Shunts can be used to treat PH and other conditions such as, but not limited to, heart failure, hypertension, kidney failure, volume overload, hypertrophic cardiomyopathy, valve regurgitation, and numerous congenital diseases. A shunt serves as a hole or small passage that allows movement of fluid from one part of a patient's body (e.g., the pulmonary artery) to another. The efficacy and safety of a shunt in its intended application largely depends on attributes such as precise shunt placement, secure shunt fixation, shunt durability, minimization of regions of possible fluid stasis, ease of deployment, and adjustability over time. As such, there is a need to constantly improve and refine shunt designs and applications to arrive at a shunt that provides safe and effective treatment while at the same time allows for ease of use and reduced costs.

### BRIEF SUMMARY OF THE INVENTION

According to a first aspect, a shunt device is manipulable between a collapsed configuration and a shunt configuration. The shunt device includes a shunt frame assembly having a first anchor and a second anchor that are configured to radially flare from the collapsed configuration toward the shunt configuration. The second anchor defines a proximal opening of the shunt device in the shunt configuration, and the first anchor and second anchor define a common central axis. The shunt device further includes a flow director that extends distally from the shunt frame assembly and defines a fluid passageway and a distal opening at an end of the fluid passageway. The flow director is configured to redirect at least some fluid flowing through the fluid passageway such that the at least some fluid is discharged through the distal opening in a fluid direction transverse to the central axis.

### BRIEF DESCRIPTION OF THE DRAWINGS

FIG. 1 is an anatomical view a human subject showing the right pulmonary artery and its position relative to other anatomical features;

FIG. 2 is a perspective view of an example shunt device in a shunt configuration;

FIG. 3 is a cross-section view of the shunt device when implanted in a human subject in the shunt configuration;

FIG. 4 shows an elastic body of the shunt device, as if it were longitudinally and flattened;

FIG. 5 schematically shows a cross-section view of the shunt device in a collapsed configuration;

FIG. 6 schematically shows a delivering step of an example method for implanting the shunt device;

FIG. 7 schematically shows a first stage of a first anchoring step of the method;

FIG. 8 schematically shows a second stage of the first anchoring step;

FIG. 9 schematically shows a second anchoring step of the method; and

FIG. 10 shows another example shunt device.

### DETAILED DESCRIPTION

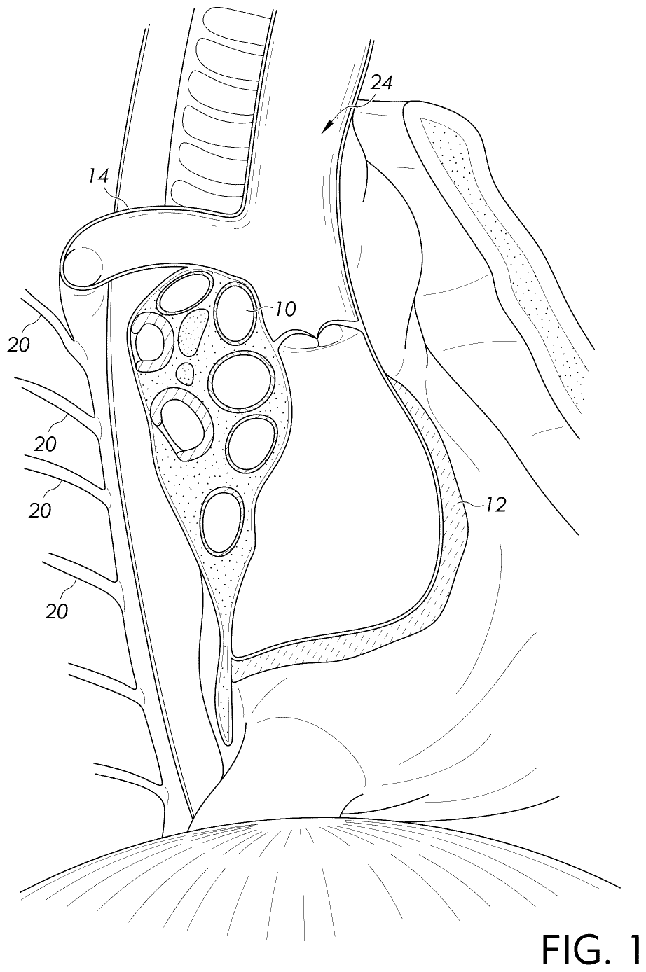

FIG. 1 is an anatomical view of a human subject showing the right pulmonary artery 10 and its position relative to other anatomical features. The right pulmonary artery 10 carries deoxygenated blood from the heart 12 to the right lung so the blood can gain oxygen and get rid of waste products like carbon dioxide. As shown in FIG. 1, a segment of the right pulmonary artery 10 extends inferior and adjacent to the azygous vein 14, which receives deoxygenated blood from the intercostal veins 20 and deposits the deoxygenated blood into the superior vena cava 24. The vena cava 24 in turn conveys the deoxygenated blood to the right atrium of the heart 12.

Pressure in the right pulmonary artery 10 can be diminished by shunting blood therefrom to another location. Shunting high-pressure blood from the right pulmonary artery 10 directly into the vena cava 24 can be undesirable because introducing high pressure into the vena cava 24 can induce or exacerbate heart-failure symptoms due to the blood immediately returning to the right side of the heart. Accordingly, it is preferable to shunt blood from the right pulmonary artery 10 into the azygous vein 14 at a location upstream of where that vessel empties into the vena cava 24. In particular, it is preferable that the blood be directed into the azygous vein 14 in a direction against its antegrade flow. This allows the high-pressure flow shunted from the pulmonary artery 10 to dissipate within the much lower-pressure azygous vein 14, including via access to the network of intercostal veins 20 in communication with the azygous vein 14; which all together encompass a large volume of low-pressure vasculature that can absorb and dissipate the relatively low flow rates of high-pressure blood shunted from the pulmonary artery 10—before the shunted blood is directed into the vena cava 24 downstream.

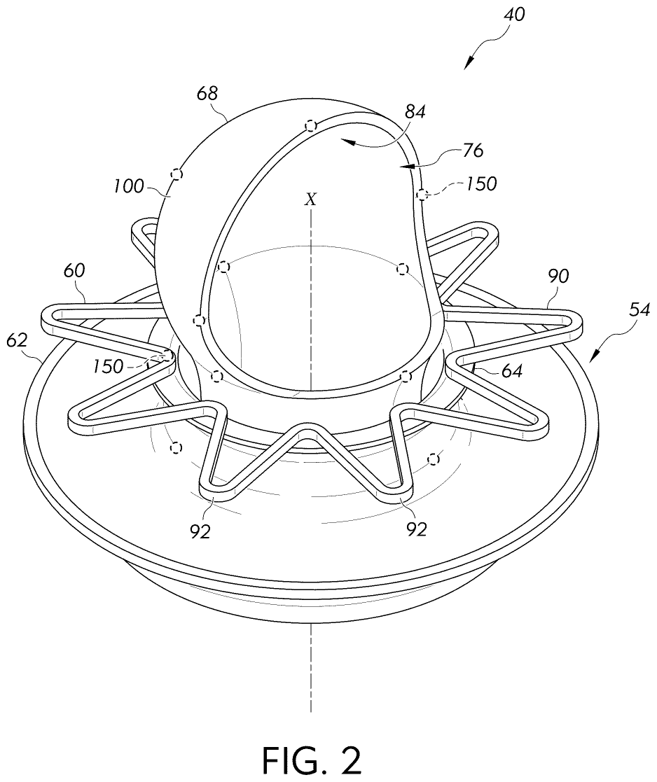

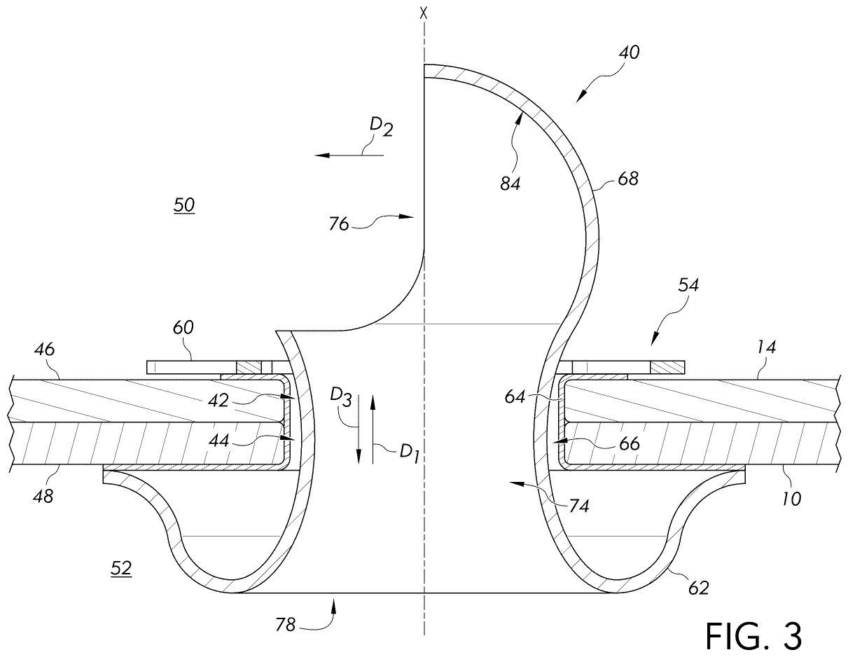

Turning to FIGS. 2 & 3, an example shunt device 40 will now be described that can be used to shunt blood from one location of an animal (e.g., human) subject's body to another. FIG. 2 shows a perspective view of the shunt device 40 by itself, while FIG. 3 shows a cross-section view of the shunt device 40 as applied to the azygous vein 14 and right pulmonary artery 10 of a human subject. In particular, respective openings 42, 44 (see FIG. 3) can be formed in the walls 46, 48 of the azygous vein 14 and right pulmonary artery 10, and the shunt device 40 can be affixed to the walls 46, 48 such that it extends through the openings 42, 44 and provides fluid communication between the respective lumens 50, 52 of the azygous vein 14 and right pulmonary artery 10.

The shunt device 40 has a shunt frame assembly 54 with first and second anchors 60, 62 that extend annularly about a common central axis X. The shunt frame assembly 54 further includes an intermediate portion 64 that extends between and is connected to the first and second anchors 60, 62. The intermediate portion 64 defines a window 66 through the shunt frame assembly 54 that is coaxial with the central axis X. Moreover, the shunt device 40 further includes a flow director 68 that extends distally from the shunt frame assembly 54 and defines a fluid passageway 74 and a distal opening 76 at an end of the passageway 74 (for the purposes of this disclosure, the terms “distal” and “proximal” when describing features of the shunt device 40 are relative to the path that fluid flows or is capable of flowing through the shunt device 40, wherein distal features are closer to the end of the flow path than proximal features).

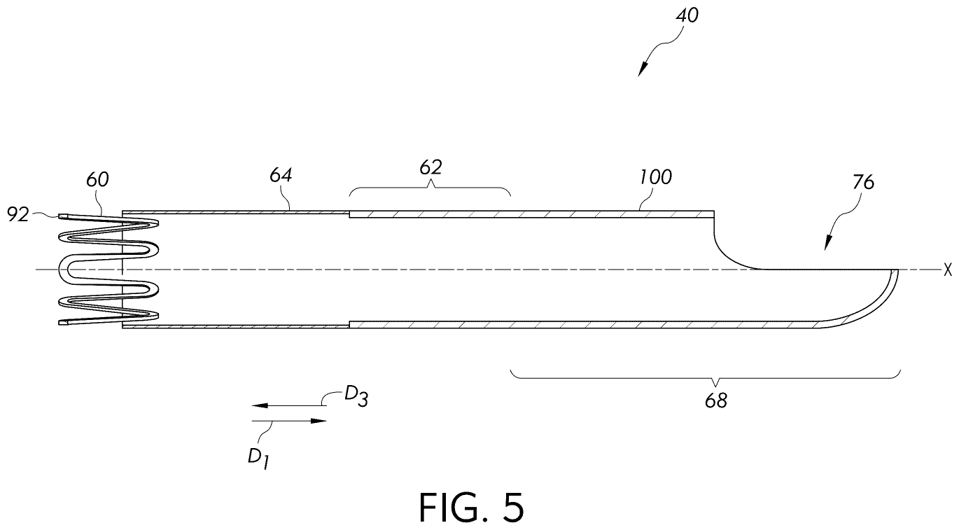

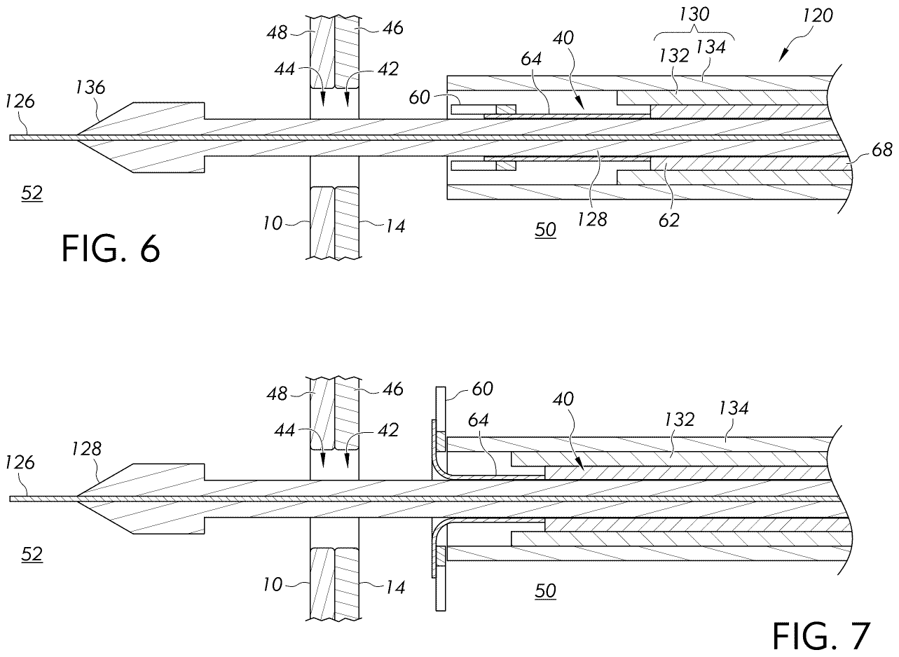

As discussed later herein, the shunt device 40 is a flexible structure that is manipulable from the configuration shown in FIGS. 2 & 3 to a collapsed configuration (see FIG. 5), which enables the shunt device 40 to be loaded into an associated delivery system 120 (see FIGS. 6-9) for implantation. During the implant process, the delivery system 120 can be operated to manipulate the shunt device 40 from its collapsed configuration back to the configuration shown in FIGS. 2 & 3, which can be referred to as a “shunt configuration” of the device 40. For now, the features of the shunt device 40 will be described with respect to their shape and arrangement in the shunt configuration.

In the shunt configuration, each of the anchors 60, 62 is a ring-like body that is radially-flared such that its outer diameter is larger in the shunt configuration as compared to the collapsed configuration (it is to be appreciated the central axis X of the shunt frame assembly 54 defines the axial and radial directions of the shunt device 40). Preferably, the outer diameters of the first and second anchors 60, 62 will be greater than the respective diameters of the openings 42, 44 in the azygous vein 14 and right pulmonary artery 10.

Moreover, the intermediate portion 64 of the shunt frame assembly 54 is an annular body comprising a flexible membrane of material (e.g., urethane foam, woven or braided fabric, expanded polytetrafluoroethylene, electrospun polyurethane, thermoplastic polyurethane, polyethylene terephthalate, polyurethane, silicone, gelatin blended nanofibrous membranes, etc.) that connects the anchors 60, 62 and can conform to (e.g., fit through) the openings 42, 44 in the azygous vein 14 and right pulmonary artery 10. At least a portion of the intermediate portion 64 can be treated with at least one therapeutic agent for eluting into a blood vessel (PA-AV), a cardiac chamber, and/or cardiac tissue. The therapeutic agent can be capable of preventing a variety of pathological conditions including, but not limited to, arrhythmias, thrombosis, systemic hypertension, pulmonary hypertension, stenosis, apoptosis, and inflammation. Accordingly, the therapeutic agent may include at least one of an anti-arrhythmic agent, anticoagulant, an antioxidant, a fibrinolytic, a steroid, an anti-apoptotic agent, an anti-overgrowth agent (i.e., capable of preventing epithelial cell overgrowth), and/or an anti-inflammatory agent. Optionally or additionally, the therapeutic agent may be capable of treating or preventing other disease or disease processes such as microbial infections and heart failure. In these instances, the therapeutic agent may include an inotropic agent, a chronotropic agent, an anti-microbial agent, and/or a biological agent such as a cell or protein. Examples of acceptable therapeutic agents include heparin, synthetic heparin analogues (e.g., fondaparinux), G(GP) II_{b}/IIIₐ inhibitors, vitronectin receptor antagonists, hirudin, antithrombin III, drotrecogin alpha; fibrinolytics such as alteplase, plasmin, lysokinase, factor XIIa, factor VIIa, prourokinase, urokinase, streptokinase; thrombocyte aggregation inhibitors such as ticlopidine, clopidogrel, abciximab, dextrans; corticosteroids such as aldlometasones, estradiols, such as 17β-estradiol, amcinonides, augmented betamethasones, beclomethasones, betamethasones, budesonides. fibrinolytic agents such as tissue plasminogen activator, streptokinase, dipyridamole, ticlopidine, clopidine, and abciximab; non-steroidal anti-inflammatory drugs such as salicyclic acid and salicyclic acid derivatives, para-aminophenol derivatives, indole and indene acetic acids (e.g., etodolac, indomethacin, and sulindac), heteroaryl acetic acids (e.g., ketorolac, diclofenac, and tolmetin) Pulmonary Artery vasodilators, prostacyclin, soluble guanylate cyclase (sGC) stimulators, Epoprostenol, Treprostinil Sodium, Selexipag, Sidenafil, Tadafil, etc.

The shunt device 40 can be implanted such that the first anchor 60 is arranged on the interior side of the vein wall 46 while the second anchor 62 is arranged on the interior side of the artery wall 48, with the intermediate portion 64 extending through the openings 42, 44 of the walls 46, 48. Preferably, the intermediate portion 64 will apply a slight tension to the anchors 60, 62 in the axial direction such that they press (directly or indirectly) against the respective walls 46, 48. Thus, a seal can be formed between each wall 46, 48 and its associated anchor 60, 62, such that fluid within the azygous vein 14 and right pulmonary artery 10 is inhibited from escaping between the walls 46, 48 and shunt frame assembly 54.

The shunt frame assembly 54 in the shunt configuration can thus anchor the shunt device 40 to the walls 46, 48 of the azygous vein 14 and right pulmonary artery 10, and establish a sealed window 66 through the walls 46, 48. Moreover, as installed, the central axis X of the shunt frame assembly 54 will be substantially perpendicular to the longitudinal axes of the azygous vein 14 and right pulmonary artery 10 at the location of the shunt device 40.

In the shunt configuration, the second anchor 62 will define a proximal opening 78 of the shunt device 40. Moreover, the flow director 68 will extend distally from the second anchor 62 through the window 66 of the shunt frame assembly 54, such that the flow director 68 extends distally past the first anchor 60 (e.g., into the lumen 50 of the azygous vein 14). Accordingly, fluid from the right pulmonary artery 10 can enter the shunt device 40 via the proximal opening 78, flow through the passageway 74 of the flow director 62, and then be discharged into the azygous vein 14 via the distal opening 76.

Notably, fluid from the right pulmonary artery 10 can flow into the fluid passageway 74 of the flow director 68 in a first direction D₁ that is substantially parallel to the frame assembly's central axis X (and substantially perpendicular to the longitudinal axes of the azygous vein 14 and right pulmonary artery 10 at the location of the shunt device 40). However, as the fluid progresses toward the distal opening 76, an inner surface 84 of the flow director 68 can redirect at least some of the fluid such that it is discharged from the distal opening 76 in a second direction D₂ transverse (e.g., substantially perpendicular) to the first direction D₁ and the central axis X. More specifically, the inner surface 84 is gradually curved such that fluid in the first direction D₁ can impinge upon the inner surface 84 and be redirected toward and through the distal opening 76 in the second direction D₂.

Preferably, the second direction D₂ will be substantially parallel but counter to the antegrade flow of blood in the azygous vein 14, although non-parallel directions that are counter to the antegrade flow are also possible. For example, an angle between the second direction D₂ and the direction of antegrade flow in the azygous vein 14 may be 45° or less, and preferably 15° or less. Put another way, an angle between the second direction D₂ and the central axis X (or the first direction D₁) can be about 45° to about 135°, and preferably about 75° to about 105°. Moreover, the distal opening 76 will preferably be located some distance (e.g., at least 1 cm, preferably at least 2 cm, most preferably at least 3 cm) away from where the azygous vein 14 empties into the vena cava 24.

In the shunt configuration, the flow director 68 of the present embodiment comprises a unique geometry that enables the redirection of flow described above. More specifically, as shown in FIG. 3, the lower end of the flow director 68 is generally tubular while the upper end has a rounded hood that defines the curved inner surface 84. However, it is to be appreciated that the flow director 68 may comprise a variety of other geometries and configurations without departing from the scope of the disclosure. For example, the upper portion of the flow director 68 may comprise an elongated tube (e.g., stent or collapsible tube) that curves and extends upstream into the lumen 50 of the azygous vein 14, such that a distal outlet of the tube discharges fluid in a direction transverse to the central axis X. Broadly speaking, the flow director 68 may comprise any configuration that defines and fluid passageway and discharges fluid in a direction transverse to the central axis X.

As noted above, the shunt device 40 is a flexible structure that can be manipulated to the collapsed configuration in FIG. 5, which enables the shunt device 40 to be loaded into a delivery system (see FIGS. 6-9) for implantation. Moreover, one or more features of the shunt device 40 can be elastically biased toward the shunt configuration, in order to facilitate manipulation of the shunt device 40 from the collapsed configuration back to the shunt configuration.

For example, as shown best in FIG. 2, the first anchor 60 comprises an annular wire body 90 having plurality of triangular peaks 92 that are circumferentially aligned about the central axis X. In the shunt configuration, each triangular peak 92 points in a radial direction relative to the central axis X. Moreover, the wire body 90 comprises a shape-memory material (e.g., Nitinol) that is flexible but elastically biased toward its configuration in FIG. 2. As a result of this design, the angle of each peak 92 can decrease to reduce the inner and outer diameters of the first anchor 60. Conversely, the angle of each peak 92 can also increase to expand the inner and outer diameters of the first anchor 60. Furthermore, the first anchor 60 can be manipulated to flip the peaks 92 such that each peak 92 points in a direction substantially parallel to the central axis (e.g., downward in FIG. 2). When the peaks are oriented in the axial direction and the angle of each peak is reduced, the first anchor 60 can have a generally tubular shape with a relatively small diameter as compared to its shape in the shunt configuration. Moreover, the first anchor 60 will be elastically biased to return to the shunt configuration from its tubular shape.

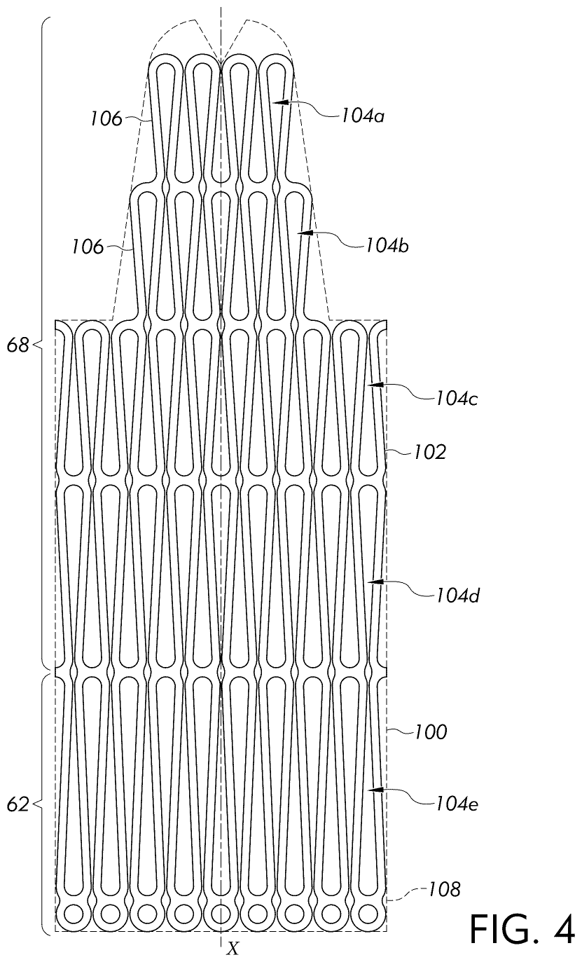

As another example, the shunt device 40 can comprise an elastic body 100 that forms the second anchor 62 and flow director 68 of the shunt device 40, and is elastically biased toward the shunt configuration of those features. FIG. 4 shows the body 100 as if it were longitudinally cut (e.g., in the axial direction) and flattened. As shown in the FIG. 4, the body 100 comprises a flexible, laser-cut frame 102 having a plurality of bands 104a-e, wherein each band 104a-e includes a plurality of loops 106 that are aligned and joined together. The body 100 further includes a flexible membrane 108 that is affixed to the frame 102 such that the membrane 108 can conform to and follow the shape of the frame 102. In the present example, the membrane 108 comprises an impermeable layer of elastic material (e.g., expanded polytetrafluoroethylene, urethane derivatives, polyethylene terephthalate, etc.) that encases the frame 102, such the frame 102 is embedded within the membrane 108. However, the membrane 108 may comprise other permeable or impermeable materials (e.g., fabric), and may be affixed to the frame 102 in alternative manners (e.g., adhesive bonding, sewing, etc.).

The left and right ends of the bands 104c-104e and membrane 108 in FIG. 4 can be joined together, such that the body 100 forms a tubular structure about the central axis X.

Moreover, the frame 102 and membrane 108 can be elastically deformed to assume the shunt configuration of the body 100 in FIG. 2. In particular, the spacing between the loops 106 in each band 104a-e can expand in areas of larger diameter, and the loops 106 can be bent to form the curvatures of the body 100 along the central axis X. Moreover, the membrane 108 will elastically deform with the frame 102 since it is affixed thereto.

Preferably, the frame 102 of the body 100 comprises a shape-memory material (e.g., Nitinol) that will be elastically biased toward the shunt configuration in FIG. 2. Accordingly, the body 100 can be manipulated from the shunt configuration to the collapsed configuration in FIG. 5, but will be elastically biased to facilitate manipulation back to the shunt configuration. Moreover, the elastic bias of the body 100 (which forms the second anchor 62 and flow director 68) can ensure that the body 100 retains its shape in the shunt configuration and maintains patency through the passageway 74.

As discussed above, the elastic body 100 forms the second anchor 62 and flow director 68 of the shunt device 40. However, it is to be appreciated that the elastic body 100 may form additional or fewer elements of the shunt device 40. For instance, in some embodiments, the elastic body 100 may form the entire shunt device 40. In such examples, the wire frame 102 would be configured such that each respective element of the shunt device 40 is elastically biased toward its shunt configuration.

Moreover, it is to be appreciated the that features of the shunt device 40 described above may comprise other shapes and configurations (e.g., materials, components, etc.) without departing from the scope of the disclosure. For example, the first and second anchors 60, 62 may comprise other shapes and configurations that can radially flare to have respective dimensions (e.g., widths or diameters) that are greater than the respective diameters of the openings 42, 44 in the azygous vein 14 and right pulmonary artery 10.

The manner in which the shunt device 40 is manipulated to assume the collapsed configuration will now be described in further detail. Specifically, from the shunt configuration in FIGS. 2 and 3, the shunt device 40 can assume the collapsed configuration in FIG. 5 by pulling the first anchor 60 in a direction D₃ away from the distal opening 76. As the first anchor 60 approaches the second anchor 62, the first anchor 60 can radially expand (e.g., via decreasing the angle between its peaks 92) and/or the second anchor 62 can radially contract (e.g., via decreasing the spacing between its loops 106) to permit the first anchor 60 to slide over and surpass the second anchor 62. The first anchor 60 can then be further pulled in the direction D₃ away from the second anchor 62 and distal opening 76 until tension is generated in the shunt device 40. As the first anchor 60 is further pulled with tension, the shunt device 40 will radially contract and axially lengthen, particularly at the first and second anchors 60, 62. Eventually, the shunt device 40 will assume the collapsed configuration in FIG. 5, such that the shunt device 40 is a generally tubular structure having the first anchor 60 at one end and the distal opening 76 at the other. Notably, the peaks 92 of the first anchor 60 will be oriented in the axial direction in the collapsed configuration.

The shunt device 40 can be returned to the shunt configuration by moving the first anchor 60 in the direction D₁ back toward the distal opening 76. As the first anchor 60 approaches the second anchor 62, the first anchor 60 can radially expand (e.g., via increasing the angle between its peaks 92) and/or the second anchor 62 can radially contract (e.g., via increasing the spacing between its loops 106) to permit the first anchor 60 to slide over and surpass the second anchor 62. The first anchor 60 can then be further moved in the direction D₁ towards the distal opening 76, and the elastic bias of the body 100 and first anchor 60 can facilitate return of the shunt device 40 to its shunt configuration.

Turning to FIGS. 6-9, an example method of implanting the shunt device 40 will now be described. FIG. 6 shows a first step of delivering the shunt device 40 to the lumen 50 of the azygous vein 14. More specifically, the shunt device 40 can be loaded into a delivery system 120 comprising a guidewire lumen 126, a dilator 128, and a sheath system 130 having an inner sheath 132 and an outer sheath 134. The inner sheath 132 extends at least partially within the outer sheath 134, the dilator 128 extends at least partially withing the inner sheath 132, and the guidewire lumen 126 that extends at least partially within the dilator 128 (and the inner and outer sheaths 132, 134). The guidewire lumen 126, dilator 128, inner sheath 132, and outer sheath 134 are translatable relative to each other, such that the dilator 128 can translate along the guidewire lumen 126, the inner sheath 132 can translate along the dilator 128, and the outer sheath 134 can translate along the inner sheath 132.

The shunt device 40 can be loaded into the delivery system 120 in its collapsed configuration such that the guidewire lumen 126 and dilator 128 extend through the shunt device 40. In particular, the shunt device 40 can be oriented on the guidewire lumen 126 and dilator 128 such that its first anchor 60 is positioned closer to the distal ends of the guidewire lumen 126 and dilator 128 than its flow director 68. Moreover, the sheath system 130 can radially confine the shunt device 40 and thus inhibit it from expanding radially toward its shunt configuration.

The delivery system 120 can be used to deliver the shunt device 40 in its collapsed configuration into the lumen 50 of the azygous vein 14. Preferably, this is a percutaneous intervention, rather than open heart surgery, which offers the benefit of being less invasive. To accomplish this, the delivery system 120 can be inserted through the jugular, radial, or femoral vein by a modified Seldinger technique, to reach the lumen 50 of the azygous vein 14 (via the vena cava 24). However, alternative pathways to the lumen 50 may be utilized in other embodiments.

Once the delivery system 120 reaches the lumen 50, a needle at the distal end of the guidewire lumen 126 can pierce the walls 46, 48 of the azygous vein 14 and right pulmonary artery 10, and a nose cone 136 at the distal end of the dilator 128 can follow therethrough to form the openings 42, 44 for the shunt device 40. Preferably, the openings 42, 44 will be located some distance (e.g., at least 1 cm, preferably at least 2 cm, most preferably at least 3 cm) away from where the azygous vein 14 empties into the vena cava 24.

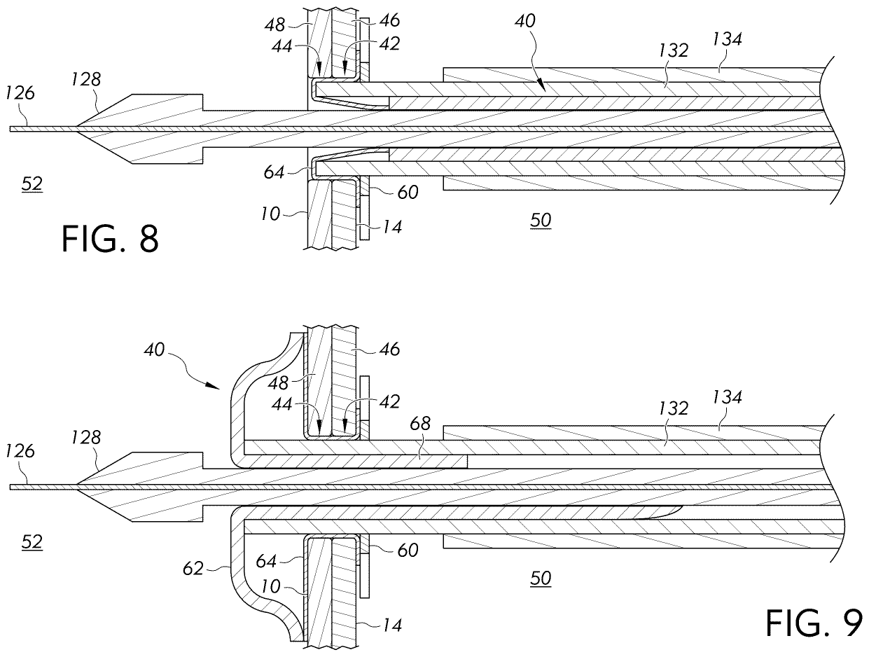

FIGS. 7 and 8 show a first anchoring step in which the first anchor 60 of the shunt device 40 is deployed and affixed to the wall 46 of the azygous vein 14. More specifically, as shown in FIG. 7, the outer sheath 134 can be operated to retract relative to the guidewire lumen 126, inner sheath 132, and shunt device 40, thereby releasing the first anchor 60 from confinement. As a result, the first anchor 60 will radially flare (due to its elastic bias) from its collapsed configuration to its shunt configuration. Then, as shown in FIG. 8, the inner sheath 132 can be operated to advance relative to the guidewire lumen 128 and outer sheath 134. During this advancement, the inner sheath 132 will engage and press against the intermediate portion 64 of the shunt device 40, causing the shunt device 40 to translate with the inner sheath 132 along the guidewire lumen 126 until the first anchor 60 presses (directly or indirectly) against the wall 46 of the azygous vein 14, thereby affixing the first anchor 60 to the wall 46.

As the inner sheath 132 further advances into and through the openings 42, 44 of the lumen walls 46, 48, the first anchor 60 will remain stationary against the wall 46 of the azygous vein 14 while the inner sheath 132 draws other portions of the shunt device 40 into and through the openings 42, 44. For example, as can be seen in FIGS. 8 and 9, the inner sheath 132 will draw the intermediate portion 64 into and through openings 42, 44, thereby inverting the intermediate portion 64 such that it surrounds the inner sheath 132 and extends from the first anchor 60 into the openings 42, 44.

Eventually, further advancement of the inner sheath 132 relative to the guidewire lumen 126 and outer sheath 134 will execute a second anchoring step. More specifically, as shown in FIG. 9, the inner sheath 132 can be advanced until the second anchor element 62 of the shunt device 40 is drawn through the openings 42, 44 of the lumen walls 46, 48 and released from confinement. As a result, the second anchor 62 will radially flare (due to its elastic bias) from its collapsed configuration to its shunt configuration. Moreover, the intermediate portion 64 will apply a slight tension to the anchors 60, 62 in the axial direction such that they press (directly or indirectly) against the respective walls 46, 48. In other words, the anchors 60, 62 will apply a compressive force to the composite wall structure of the lumen walls 46, 48, thereby anchoring the shunt device 40 to the lumen walls 46, 48.

Finally, the inner sheath 132 and the guidewire lumen 126 can be retracted relative to the shunt device 40 to complete the implant process. That is, the inner sheath 132 can be retracted to release the flow director 68 from its confinement, allowing the inner sheath 132 (and its inner diameter) to radially expand. The flow director 68 will then radially flare (due to its elastic bias) from its collapsed configuration to its shunt configuration. Moreover, the guidewire lumen 126 can be retracted through the flow director 68 until it completely exits the shunt device 40. As a result, the shunt device 40 will assume its final configuration as described above with respect to FIG. 3.

The shunt device 40 and its method of implant described above can be used to treat PH, since the shunt device 40 as implanted can shunt blood from the right pulmonary artery 10 to the azygous vein 14. In particular, pressure in the right pulmonary artery 10 is diminished by shunting blood therefrom into the azygous vein 14 upstream from where it intersects (and terminates at) the superior vena cava 24. In this manner, the shunt device 40 can accommodate and redirect blood flow from (thereby relieving pressure within) the right pulmonary artery 10, into the azygous vein 14 along a counter-current and substantially coaxial path therein, where pressure is lower. Thus, the azygous vein 14 and its network of upstream vessels can absorb and dissipate excess pressure from the pulmonary artery 10 with minimal adverse effects. Unloading the pulmonary artery pressure by the shunt device 40 consequently decreases patient clinical symptoms of pulmonary hypertension and heart failure.

However, it is to be appreciated that the shunt device 40 and its method of implant may be used to treat other conditions besides PH. Indeed, the shunt device 40 may be similarly implanted to shunt blood to and/or from different areas of the body. In some examples, the shunt device 40 may be used to shunt blood between two body lumens that are separated by a common wall. In other examples, the shunt device 40 may be used to shunt blood between two vessels having separate walls that are spaced apart from each other. In such examples, the intermediate portion 64 may be configured to radially flare in the shunt configuration such that it functions as a spacer between the walls.

Moreover, the shunt device 40 may incorporate features such as radio opaque markers, or intelligent sensors that can monitor key metrics such as pressure and flow in real time during operation and allow for the optimization of such metrics. For example, as shown schematically in FIG. 2, the shunt device 40 can include a plurality of radio opaque markers 150 provided on (e.g., formed with or attached to) the flow director 68. One or more markers 150 may be provided at a distal end of the flow director 68, or somewhere between its proximal and distal ends. In some examples, a set of markers 150 may be aligned circumferentially about the flow director 68, wherein an angular distance (relative to the central axis X) between adjacent markers 150 of the set is about 50° to 70° (preferably about 60°), or about 80° to 100° (preferably about 90°). Moreover, in some examples, one or more markers 150 may be provided on an inner location of the first anchor 60 between adjacent peaks 92. Such locations of the markers 50 can be useful to determine if the shunt device 40 has properly expanded to its shunt configuration.

As another example, a pressure sensor can be affixed to the proximal end of the shunt device 40 and delivered towards the patient's heart. Moreover, the sensor can be foldable to collapse with the shunt device 40. The pressure sensor can provide pressure related data by use of an external measuring device. A variety of excitation systems, such as a transmitting antenna, can be electromagnetically coupled to the sensor to communicate pressure data from the sensor to an analyzer that can be used in conjunction with an interface module to be used in real time by physicians. A current can be induced in the sensor, which oscillates at the resonant frequency of the sensor. This oscillation causes a change in the frequency spectrum of the transmitted signal. From this change, the bandwidth and resonant frequency of the particular sensor may be determined by an impedance system, from which the corresponding change in pressure can be calculated.

In addition or alternatively, a flow sensor can be incorporated in a distal end of the flow director 68. The flow sensor can likewise provide information relating to the flow of blood within a patient that can optimize use of the shunt device 40. Knowing the flow of the fluid within the shunt device 40 will allow for prediction of its behavior through mathematical formulas.

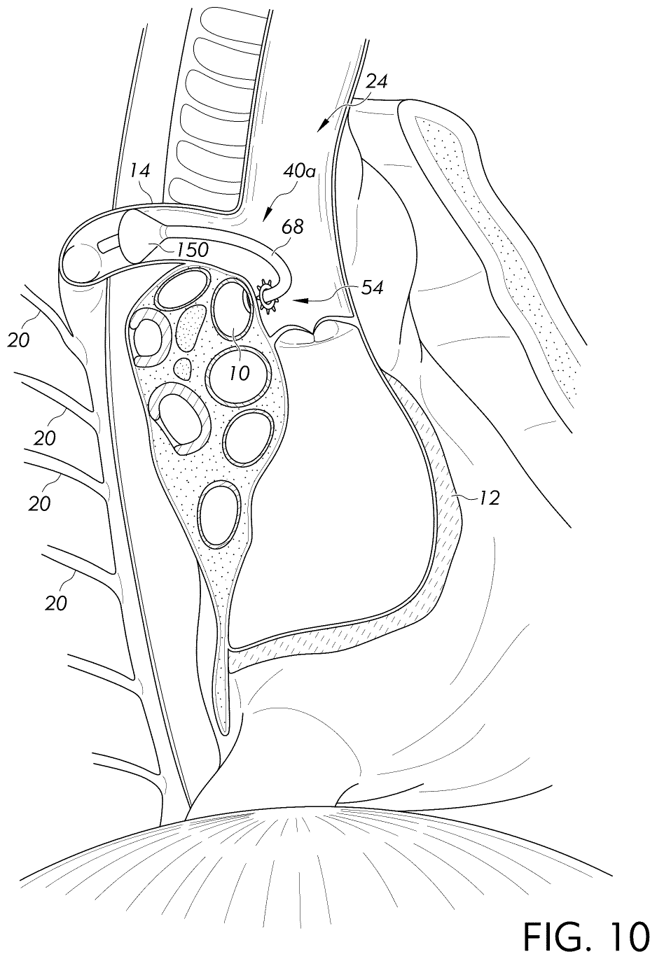

FIG. 10 illustrates another embodiment of the shunt device 40a that connects the pulmonary artery to the superior vena cava. In this embodiment, the shunt device 40a is anchored to the walls of the right pulmonary artery 10 and the vena cava 24 to provide a window therethrough. Moreover, the flow director 68 is a stent (e.g., a covered stent) that proceeds from the shunt frame assembly 54 in the superior vena cava 24, and then into the azygous vein 14. At the distal end of the flow director 68, a cone 150 is disposed coaxially around the flow director 68. The wide end of the cone 150 is disposed towards the distal end of the flow director 68, while at the opposite end the cone 150 is fitted to the flow director 68. The cone 150 serves as an anchor to fixate the distal opening 72 of the flow director 68 within the azygous vein 14, and also helps to fix its longitudinal location upstream within the azygous vein 14 relative to where it empties into the superior via cava 24. The cone 150 could act as a way to center the distal opening 76 of the flow director 68 substantially centrally within the azygous vein 14. Furthermore, the cone 150 could be fitted at the most distal end of the flow director 68. The cone 150 may be made of a porous mesh material that allows blood to pass through, for instance a bare metal frame. Other porous materials that allow the passage of blood may be adequate substitutes. Alternatively, the cone 150 element may be provided as a simple wire-based structure (e.g. nitinol wire), wherein opposing wire segments hold the cone 150 in place relative to the flow director 68 and together therewith present a substantially trapezoidal shape when viewed from the side.

Moreover, the flow director 68 in FIG. 10 has a venturi configuration, with the distal opening 72 having a smaller diameter than the passageway of the flow director 68 at its proximal end. This allows the device to take advantage of the Bernoulli principle of conservation of energy to reduce pressure (and increase flow velocity) of blood that is shunted from the pulmonary artery 14 and delivered to the azygous vein 14. These effects serve dual purposes of a) reducing the pressure of shunted blood before it is delivered to the azygous vein 14, in order to diminish the degree of dissipation/absorption required within the azygous/intercostal venous network to accommodate the shunted blood, and b) increasing the velocity of the shunted blood as it enters the azygous vein 14 in a counter-current flow direction via the distal opening 72 of the flow director 68 in order to reach and have access to a greater depth of the azygous/intercostal venous system for dissipating its pressure prior to being redirected back into the vena cava 14.

Still further, it is to be appreciated that other types of delivery systems and methodology may be used to implant the shunt device 40 without departing from the scope of the disclosure. For example, the sheath system 130 of the delivery system 120 described above comprises inner and outer sheaths 132, 134 that are translatable relative to each other. In other examples, the sheath system 130 may comprise a single sheath that confines the shunt device 40 and translates relative to the guidewire lumen 130 to sequentially release portions of the shunt device 40 in the manner described above. Other example methods for implanting the shunt device 40 or other shunts are briefly described below.

One example method of retrograde implantation comprises: 1) accessing a wire to the right atrium (RA) from the internal jugular, radial, or femoral vein by modified Seldinger technique; advancing the wire across the tricuspid and pulmonary valves to reach the right pulmonary artery (RPA); 2) advancing a second wire through the femoral vein by modified Seldinger technique and advancing it to the superior vena cava (SVC) towards the azygous vein (AZV); 3) advancing a catheter and a needle or wire to cross the inferior AZV wall to reach the RPA anterior wall and cross it by fluoroscopic and echocardiographic guidance; 4) advancing a wire into the main PA towards the left PA and advancing a dilator catheter through it; 5) exchanging the second wire for a snare wire inside the PA, grabbing the first RPA wire and pulling it across the RPA-AZV wall, and then advancing a dilator catheter through it inside the AZV; 6) advancing the RPA/AZV shunt through it until it reaches the AZV; 7) retracting the delivery sheath until the distal portion of the RPA/AZV shunt is released inside the AZV; 8) pulling back the RPA/AZV stent-shunt until the AZV side of the shunt is anchored or attached to the inferior AZV wall; and 9) deploying the RPA side of the stent-shunt and securing it in place by pulling the delivery sheath, thereby removing the delivery system, wherein the RPA/AZV shunt blood flow may be assessed and quantified by echocardiography and the PA Pressures changes may be measured by Swan Ganz catheter.

Another example method of antegrade implantation can comprise: 1) accessing a wire to the RA from the internal jugular, radial, or femoral vein by modified Seldinger technique; 2) advancing the wire across the tricuspid and pulmonary valves to reach the RPA; 3) advancing a needle or wire across the anterior RPA wall to reach the inferior wall of the AZV, and crossing the needle or wire by fluoroscopic and echocardiographic guidance; 4) advancing a wire into the AZV and advancing a dilator catheter through it, following a delivery sheath with a collapsible RPA/AZV stent-shunt into the AZV; 5) retracting the delivery sheath until the RPA/AZV stent-shunt is released inside the AZV, and pulling it back to anchor the stent-shunt to the AZV wall; and 6) pulling the delivery sheath back, releasing the RPA side of the RPA/AZV stent-shunt and securing it in place, thereby removing the delivery system, wherein the RPA/AZV Stent-Shunt blood flow may be assessed and quantified by echocardiography and the PA Pressures changes may be measured by Swan Ganz catheter.

Another example method of retrograde implantation can comprise: 1) accessing a wire to the RA from the internal jugular, radial, or femoral vein by modified Seldinger technique; 2) advancing the wire across the tricuspid and pulmonary valves to reach the RPA; 3) advancing a second wire through the femoral vein by modified Seldinger technique and advancing it to the SVC; 4) advancing a catheter and a needle or wire to across the posterior SVC wall to reach the RPA anterior wall and crossing it by fluoroscopic and echocardiographic guidance; 5) advancing a wire into the main PA towards the left PA and advancing a dilator catheter through; 6) exchanging the second wire for a snare wire inside the PA, grabbing the first RPA wire and pulling it across the RPA-SVC wall; 7) advancing a dilator catheter through the second wire and directing it through the catheter inside the AZV; 8) advancing the RPA/AZV shunt through it until it reaches the AZV; 9) retracting the delivery sheath until the distal portion of the RPA/AZV shunt is released inside the AZV; 10) pulling back the RPA/AZV shunt until the SVC side of the shunt is anchored or attached to the posterior SVC wall; and 11) deploying the RPA side of the shunt and securing it in place by pulling the delivery sheath, thereby removing the delivery system, wherein the RPA/AZV Stent-Shunt blood flow may be assessed and quantified by echocardiography and the PA Pressures changes may be measured by Swan Ganz catheter.

Another example method of antegrade implantation can comprise: 1) accessing a wire to the RA from the internal jugular, radial, or femoral vein by modified Seldinger technique; 2) advancing the wire across the tricuspid and pulmonary valves to reach the RPA; 3) advancing a needle or wire across the anterior Right Pulmonary Artery (RPA) wall to reach the posterior wall of the SVC, and crossing the needle by fluoroscopic and echocardiographic guidance; 4) advancing a wire into the SVC towards the Azygous Vein (AZV) and advancing a dilator catheter through it, following a delivery sheath with a collapsible RPA/AZV shunt into the AZV; 5) retracting the delivery sheath until the RPA/AZV shunt is released inside the AZV, and pulling it back to anchor the shunt to the SVC wall; and 6) pulling the delivery sheath back, releasing the RPA side of the RPA/AZV shunt and securing it in place, thereby removing the delivery system, wherein the RPA/AZV Stent-Shunt blood flow may be assessed and quantified by echocardiography and the PA Pressures changes may be measured by Swan Ganz catheter.

The invention has been described with reference to the example embodiments described above. Modifications and alterations will occur to others upon a reading and understanding of this specification. Example embodiments incorporating one or more aspects of the invention are intended to include all such modifications and alterations insofar as they come within the scope of the appended claims.

1. A shunt device that is manipulable between a collapsed configuration and a shunt configuration, the shunt device comprising:
a shunt frame assembly that includes:
a first anchor at a first end of the shunt frame assembly that is configured to flare from the collapsed configuration toward the shunt configuration, and
a second anchor at a second end of the shunt frame assembly that is configured to flare from the collapsed configuration toward the shunt configuration, wherein the second anchor defines a proximal opening of the shunt device in the shunt configuration,
wherein the first anchor and second anchor define a common central axis; and
a flow director that extends distally from the shunt frame assembly and defines a fluid passageway and a distal opening at an end of the fluid passageway,
wherein in the shunt configuration, the shunt device is configured to receive fluid through the proximal opening, convey the fluid through the fluid passageway, and discharge the fluid through the distal opening, and
wherein the flow director is configured to redirect at least some fluid flowing through the fluid passageway such that the at least some fluid is discharged through the distal opening in a fluid direction transverse to the central axis.

2. The shunt device according to claim 1, wherein the first anchor and second anchor are elastically biased to flare from the collapsed configuration.

3. The shunt device according to claim 1, wherein the first anchor comprises an annular body having a plurality of anchor elements circumferentially aligned about the central axis.

4. The shunt device according to claim 3, wherein the anchor elements are pointed radially in the shunt configuration, and axially in the collapsed configuration.

5. The shunt device according to claim 1, wherein the shunt device comprises an elastic body that forms the second anchor portion and the flow director, wherein the elastic body comprises a wire frame and a membrane affixed to the wire frame.

6. The shunt device according to claim 5, wherein the wire frame is embedded within the membrane.

7. The shunt device according to claim 1, wherein the shunt frame assembly includes an intermediate portion that extends between and is connected to the first anchor and second anchor, wherein the intermediate portion defines a window that is coaxial with the central axis.

8. The shunt device according to claim 7, wherein in the shunt configuration, the flow director extends distally from the second anchor through the window of the intermediate portion, such that the flow director extends distally past the first anchor.

9. The shunt device according to claim 1, wherein an angle between the fluid direction and the central axis is about 45° to about 135°.

10. A method of implanting the shunt device accordingly to claim 1 to provide fluid communication between a first body lumen and a second body lumen separated by a wall structure, the method comprising:
a delivering step that comprises delivering the shunt device to the first body lumen in the collapsed configuration;
a first anchoring step that comprises flaring the first anchor within the first body lumen; and
a second anchoring step that comprises flaring the second anchor within the second body lumen, such that the first anchor and second anchor affix the shunt device to the wall structure,
wherein the shunt device assumes the shunt configuration to provide fluid communication between the first body lumen and second body lumen.

11. The method according to claim 10, wherein the delivering step includes delivering the shunt device with a delivery system comprising a guidewire lumen and a sheath system, wherein the guidewire lumen extends through the shunt device, and the sheath system radially confines the shunt device in the collapsed configuration.

12. The method according to claim 10, wherein the first anchoring step comprises operating the sheath system to release the first anchor.

13. The method according to claim 12, wherein the second anchoring step comprises operating the sheath system to release the second anchor.

14. The method according to claim 10, wherein the method comprises forming an opening in the wall structure prior to the first anchoring step.

15. The method according to claim 10, wherein the wall structure comprises a first wall that defines the first body lumen, and a second wall that defines the second body lumen.

16. The method according to claim 10, wherein after the second anchoring step, the first anchor and second anchor apply compressive force to the wall structure.

17. The method according to claim 10, wherein the shunt frame assembly includes an intermediate portion that extends between and is connected to the first anchor and second anchor, wherein the intermediate portion applies tension to the first anchor and second anchor.

18. The method according to claim 10, wherein in the shunt configuration:
the shunt device receives fluid through the proximal opening, conveys the fluid through the fluid passageway, and discharges the fluid through the distal opening, and
the flow director redirects at least some fluid flowing through the fluid passageway such that the at least some fluid is discharged through the distal opening in the fluid direction transverse to the central axis.

19. The method according to claim 10, wherein:
the first body lumen is a lumen of an azygous vein of the subject, and
the second body lumen is a lumen of a right pulmonary artery of the subject.

20. The method according to claim 19, wherein in the shunt configuration, the distal opening of the shunt device is located at least 1 cm from where the azygous vein intersects with a superior vena cava of the subject.
